(19) United States
(12) Patent Application Publication
(10) Pub. No.: US 2023/0404481 A1
(43) Pub. Date: Dec. 21, 2023

(54) SHIELDED HELMET TO IMPROVE SENSING OF ELECTROMAGNETIC FIELDS

(71) Applicants: Dan Miulli, Riverside, CA (US); James Garvin Wiginton, IV, Riverside, CA (US); James Brazdzionis, Redlands, CA (US); Paras Savla, Riverside, CA (US)

(72) Inventors: Dan Miulli, Riverside, CA (US); James Garvin Wiginton, IV, Riverside, CA (US); James Brazdzionis, Redlands, CA (US); Paras Savla, Riverside, CA (US)

(21) Appl. No.: 18/149,980

(22) Filed: Jan. 4, 2023

Related U.S. Application Data
(60) Provisional application No. 63/365,051, filed on May 20, 2022.

(51) Int. Cl. A61B 5/00; H05K 9/00; A61B 5/291

(52) U.S. Cl. CPC A61B 5/6803; H05K 9/0049; H05K 9/006; A61B 2562/227; A61B 2562/0209; A61B 2562/182; A61B 5/291

(57) ABSTRACT

A device for shielding electromagnetic fields is provided. The device embodies a helmet having layers of a nickel-iron ferromagnetic alloy, a copper mesh, and an air gap; at least one channel extending radially from the helmet; at least one plug configured to removably fit into respective channels wherein each plug is operative shielding electromagnetic fields by way of the channel; and at least one sensor incorporated into the helmet.

### CROSS-REFERENCE TO RELATED APPLICATION

This application claims the benefit of priority of U.S. provisional application No. 63/365,051, filed May 20, 2022, the contents of which are herein incorporated by reference.

### BACKGROUND OF THE INVENTION

The present invention relates to electromagnetic fields and, more particularly, to a shielded helmet to improve sensing thereof.

Real time measurements of brain activities are traditionally difficult to measure. Magnetoencephalography has been utilized to measure the generated electromagnetic field of the brain during neuronal activity. Magnetoencephalography is reliant on sensors to measure the electromagnetic field as well as shielding to exclude the electromagnetic field generated by objects within the surrounding environment. Typically, this measurement is enhanced with superconducting materials and shielded in the form of a large scale electromagnetic shielded room. This has large investment costs regarding super-conduction, space requirements, and financial investment. There is additionally a lack of portability regarding needing the creation of a fixed room to appropriately exclude external interference.

Electromagnetic fields are generated by a variety of structures. For example, in medical fields, neurons generate an electrochemical gradient and transmit signals to other local neurons. These neurons simultaneously fire in a summative fashion to generate an electromagnetic field. This electromagnetic field has been found to be measured through a variety of sensor modalities which may involve super conducting quantum interference devices (SQUIDs), optically pumped magnetometers (OPM) or induction sensors. These sensors have been utilized in devices for magnetoencephalography which acts as an imaging modality in neural sciences. These devices all rely on shielding to exclude higher strength electromagnetic fields which may mask the smaller strength electromagnetic field generated by the brain.

Devices designed to measure the electromagnetic field of the brain are typically reliant on SQUID devices that require supercooling and super conduction. These devices then require extensive shielding in the form of shielded rooms to mitigate external interference and isolate targeted electromagnetic fields. Other devices have been constructed for measurement that utilize alternative technologies including optical pumped magnetometers and induction sensors. However, these devices additionally all require the usage of complex shielding.

Electromagnetic field sensors are reliant on large scale, shielded rooms to shield from external electromagnetic fields, and additional shielding or super conduction to improve measurement. These shielded rooms require large financial investments and space investments that limit the ability for research and development as well as for clinical usage. Hospitals and research companies have limited space and budgets which reduce the ability for magnetoencephalography and other technologies that rely on large, shielded rooms. Additionally, even when constructed these shielded rooms require a subject to be moved to the shielded room.

As can be seen, there is a need for a device a compact and affordable device that enables the sensing of electromagnetic fields.

### SUMMARY OF THE INVENTION

In one aspect of the present invention, a device for shielding of electromagnetic fields includes the following: a helmet comprising layers of a nickel-iron ferromagnetic alloy, a copper mesh, and an air gap; at least one channel extending radially from the helmet; at least one plug configured to removably fit into respective channels wherein each plug is operative shielding electromagnetic fields by way of the channel; and at least one sensor incorporated into the helmet.

In another aspect of the present invention, the above-mentioned device further includes wherein the helmet operates by a method including the following: providing shielding from an external electromagnetic field; containing an electromagnetic field on an interior of the helmet; and funneling the electromagnetic field on the interior of the helmet to the at least one sensor.

In yet another aspect of the present invention, the above-mentioned method further includes: identifying generated electromagnetic fields by the at least one sensor; operatively associating the at least one plug into the at least one channel, respectively, to shield electromagnetic fields travelling through said channel, wherein the at least one sensor is incorporated into the at least one channel, wherein each plug is composed of layers of a nickel-iron ferromagnetic alloy, a copper mesh, and an air gap, and wherein the helmet is configured to fit on a user's head.

Advantageously, the present invention results in lesser space requirements and portability.

As the shielding is localized to a helmet, costs related to materials are reduced as less material is necessary to construct a shielded helmet than construct a large scale shielded room.

These and other features, aspects and advantages of the present invention will become better understood with reference to the following drawings, description, and claims.

### BRIEF DESCRIPTION OF THE DRAWINGS

FIG. 1 is a front perspective view of a helmet according to an embodiment of the present invention with a user wearing the helmet;

FIG. 2 is a front view thereof;

FIG. 3 is a bottom view thereof; and

FIG. 4 is a flow chart describing a process of use according to an embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

A general overview of the various features of the invention will be provided, with a detailed description following. Broadly, an embodiment of the present invention provides a method and device blocking, stopping, channeling, and/or funneling electromagnetic fields. The device may be a helmet which holds a sensor, improves measurement, and provides appropriate shielding to exclude an external electromagnetic field on an exterior of the helmet and contain a generated electromagnetic field of the subject on an interior of the helmet and funnels said field to sensors.

A modular approach may be utilized to allow for sensors to be placed and directed at targeted areas of interest.

In some embodiments of the present invention, a nickel-iron ferromagnetic alloy, such as Mu-metal®, and a copper mesh form a shielded helmet that provides adequate and appropriate shielding, improves measurements, and isolates that shielding to a lightweight helmet that increases portability and eliminates the need to create a fixed space in the form of a shielded room. The description herein repeatedly refers to Mu-metal®. It is noted that the present invention is not particularly limited to Mu-metal®. When Mu-metal® is discussed, any proper substitute may be utilized.

In some embodiments of the present invention, a helmet provides effective shielding but limits the size of the shielding to that of the approximate size of the subject of interest. This helmet shape and size allow for portability, which allows for transportation of the sensors and helmet to the subject. The helmet is reduced in size compared to the size of a shielded room which allows for usage of fewer materials and reduces costs. Furthermore, as no shielded room is required, it allows for improved space optimization for research and clinical settings where magnetoencephalography or alternative electromagnetic field sensing may be utilized. Further the present invention may utilize a modular approach, allowing for electromagnetic field sensors and channels to be placed in different areas of interest on the helmet to measure different regions of the brain active in different activities without the use of super conduction.

A layered design of the present invention may provide a structural component to block external electromagnetic fields from sensors within or passing through the layered design. This multilayer design has been demonstrated to effectively exclude external electromagnetic field noise and allow for appropriate measurements of the generated electromagnetic field created by the brain in human subjects participating in multiple activities. An air gap may be added to the electromagnetic field insulation between the two sets of Mu-metal® and copper layers. The Mu-metal® attenuates the magnetic field, and the copper mesh layers attenuate the electric field. By shaping all of these layers into a helmet, this invention provides a structure to place on a subject for measurement of an electromagnetic field. Cutouts and subsequent EMF channel insertion allow for propagation of an electromagnetic field up the channel and housing for subsequent sensing.

This device may be utilized in a clinical setting like magnetoencephalography. Using currently available sensors already marketed that are functional in measuring the electromagnetic field, this helmet would be placed around a subject. Sensors would be placed within the electromagnetic field housing which would be subsequently placed into the electromagnetic field channel attached to the helmet with all non-utilized spaces plugged using shielded plugs. With the helmet in place around the subject, interference from nearby devices and baseline electromagnetic fields are appropriately shielded while allowing propagation through the channels and housing. This allows for the electromagnetic field sensors to identify and/or measure generated electromagnetic fields. This would allow for usage in diagnostics (such as to diagnose seizure activity or epilepsy), identify changes related to structural abnormalities within a brain (for example tumors), as well as to identify regions of the brain that are more active than others which is valuable in neuroscience research to increase the understanding of cortical function. The portability of this device allows for usage in subjects that may have decreased mobility. The reduction of the shielding to a helmet in comparison to a shielded room reduces costs requirements for investment and research and may increase utilization throughout multiple hospital systems or research facilities by eliminating the space requirements for shielded rooms. It is also customizable with multiple holes or apertures for sensor placement that may be plugged in a modular fashion to preserve shielding integrity. This modular approach allows for intersections of regions of measurement to allow for localization to identify active regions and allows for the researcher or clinician to target the region they wish to measure more effectively.

A copper mesh and Mu-metal® layers provide shielding for the present invention. An air gap provides a buffer between the two layers. Plastic layers provide structure for the layers, but the specifics of plastic type and thickness are not critical to the function as the plastic does not receive or modify the electromagnetic fields. A plastic tubular layer may secure the sensor to the helmet. A housing Mu-metal® layer may block external electromagnetic fields and propagate the internal measured electromagnetic field. A cutout in the helmet may form a space for the placement of an electromagnetic field channel and housing for a sensor. The plastic tubular layer and housing Mu-metal® layer may form a housing for the sensor. A plurality of housings may be dictated by a number of sensors being used simultaneously. An external plastic disc layer of the plug and internal plastic disc layer may add structural integrity. The external copper mesh layer, external Mu-metal® disc layer, air gap, internal Mu-metal disc, internal copper mesh layer, Mu-metal® tubular layer, and a combination thereof may provide shielding. An external plastic cap for the plug provides proper support with and securement to the helmet and channel. A plurality of plugs is dictated by a number of cutouts in the helmet where a sensor and sensor housing are not placed. The plugs may be configured to removably fit into respective channels and be operative for providing shielding from electromagnetic fields travelling by way of the channels.

An internal plastic layer may provide an inner layer to protect from a surface of a subject being studied. It also may provide a framework for the first copper mesh layer to fit between the first Mu-metal® layer. The inner plastic layer may measure approximately 0.25 inches. Potential space for copper mesh may measure approximately 0.25 inches. The copper mesh lies between the inner plastic layer and the inner Mu-metal® layer. The Mu-metal® layer may be approximately 0.014 inches and lies between the copper mesh layer and the air gap. The air gap may be approximately 1 inch. The second Mu-metal® layer lies between the air gap and the second copper mesh layer. The second Mu-metal® layer measures approximately 0.014 inches and the second copper mesh layer measures approximately 0.25 inches. An external layer of Mu-metal® may be welded to the inner layer of Mu-metal® and outer layer of Mu-metal® to provide structural integrity. The outer plastic layer is after the copper mesh layer and measures approximately 0.25 inches and may be annealed to the inner plastic layer. The space cutout is a hole or holes that are cut out of the helmet at variable intervals to maintain the space for sensor placement. The cutouts involve the external plastic, copper mesh, Mu-metal® inner and exterior layers, air gap, and copper mesh layers. The inner plastic layer may be spared from being cut out. This cutout allows for insertion of the channel. For the channel, the internal layer of Mu-metal® measures approximately 0.014 inches in thickness and is wrapped by a layer of interlaced copper mesh. The copper mesh may be surrounded by an external layer plastic. The Mu-metal® from the channel may be soldered to the Mu-metal® layers to provide structural integrity as well as integrity in shielding. The copper mesh may be continuous with the outer layer and subsequently inner layers of copper mesh.

A plastic tubular layer may be formed in a tube with the inner diameter accommodated to fit a size of an electromagnetic field sensor. The housing Mu-metal® layer may be wrapped around the plastic tubular layer and secured with adhesive. The combined housing Mu-metal® layer and plastic tubular layer has an approximate combined diameter the size of the inner diameter of the channel composed of the external layer of plastic forming channel, internal layer of interlaced copper mesh in channel, and internal layer of Mu-metal® in channel. The combined housing Mu-metal® layer and plastic tubular layer slides into the combined channel layers. A separate plug may fit within the cutouts. The plug may be composed of an external plastic disc layer which is layered on top of an external copper mesh disc for the plug and external Mu-metal® disc for the plug and held together with adhesive. The air gap measuring approximately 1 inch thick provides a gap between the external Mu-metal® disc for the plug and the internal Mu-metal® disc for the plug. The internal Mu-metal® disc for the plug may be layered on top of an internal copper mesh disc for the plug and inner plastic disc layer. An outer Mu-metal® tubular layer may wrap around the side of the combined external plastic disc just under the plastic and abutting it, the external copper mesh disc, external Mu-metal® disc, air gap, internal Mu-metal disc, and copper mesh disc and inner plastic layer. This outer Mu-metal® tubular layer may be soldered to the internal Mu-metal® disc and external Mu-metal® disc for structural support. An external plastic cap for the plug may be secured on top of the external plastic disc to provide a rim of surface to keep the plug from slipping into the electromagnetic field channel layers. A copper wire may be connected to the inner copper layer and will provide as a ground on the patient.

The inner plastic layer may be molded to the desired size and shape. It may be molded into the shape of a helmet to cover a subject's head. A minimum size may allow for adequate sensor placement through the cutouts at the bases of the subject's head. The interlaced copper mesh layer may be laid on top of the inner plastic layer in the same shape as the inner plastic layer. The interlaced copper may be in a space with a predetermined thickness. That thickness may be approximately 1 inch. There may be multiple layers of copper mesh. The inner Mu-metal® layer may be then molded to the same shape as the to form a layer on top of the inner plastic layer. A proportionally sized outer Mu-metal® layer may be formed allowing for layering on top of the inner Mu-metal® layer while leaving a space of approximately 1 inch to provide an air gap. A layer of interlaced copper mesh to form the outer layer of interlaced copper mesh is layered over the outer Mu-metal® layer. An external Mu-metal® layer may be formed measuring approximately 0.014 inches in thickness and is welded to the inner Mu-metal® and outer Mu-metal® layers to preserve structural integrity and maintain the air gap. This external Mu-metal® layer may be placed on the sides of the layered sheets to form the edges of the desired helmet shape leaving the inner plastic layer as the internal but exposed layer on the bottom, the external Mu-metal® layer exposed on the lateral edges and then at this period in manufacturing the external interlaced copper mesh layer exposed superiorly. An outer plastic layer in a predetermined shape may be molded and placed over the outer copper mesh layer. The outer plastic layer and inner plastic layer may be adhered together to cover the external Mu-metal® layer. Cutout(s) may be made in the helmet at the desired locations for where sensors will be placed.

The internal layer of Mu-metal® measures approximately 0.014 inches in thickness and may be wrapped inside a layer of interlaced copper mesh. The copper mesh may wrap inside and around an external layer plastic. The entire channel may be inserted. The Mu-metal® from the channel may be soldered to the Mu-metal® layers to provide structural integrity as well as integrity in shielding. This channel abuts the internal plastic layer. The helmet device as a whole then surrounds the substance that generates the electromagnetic field. The combined housing Mu-metal® layer and plastic tubular layer slide into the combined channel layers. A separate plug may be placed within the cutouts. The plug may be composed of an external plastic disc layer which is layered on top of an external copper mesh disc for the plug and external Mu-metal® disc for the plug and held together with adhesive. The air gap provides a gap between the external Mu-metal® disc for the plug and the internal Mu-metal® disc for the plug. The internal Mu-metal® disc for the plug may be layered on top of an internal copper mesh disc for the plug and inner plastic disc layer. An outer Mu-metal® tubular layer may wrap around the side of the combined external plastic disc just under the plastic and abutting it, the external copper mesh disc, external Mu-metal® disc, air gap, internal Mu-metal disc and copper mesh disc and inner plastic layer. This outer Mu-metal® tubular layer may be soldered to the internal Mu-metal® disc and external Mu-metal® disc for structural support. An external plastic cap for the plug is secured on top of the external plastic disc to provide a rim of surface to keep the plug from slipping into the electromagnetic field channel layers.

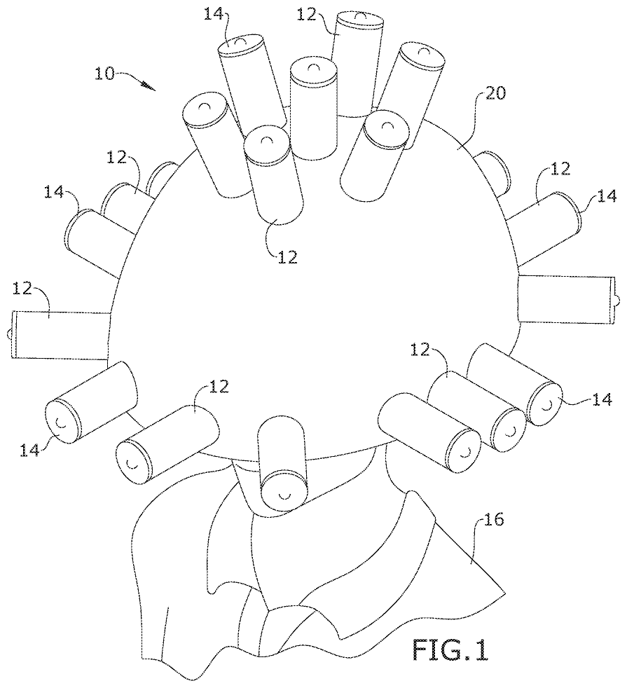

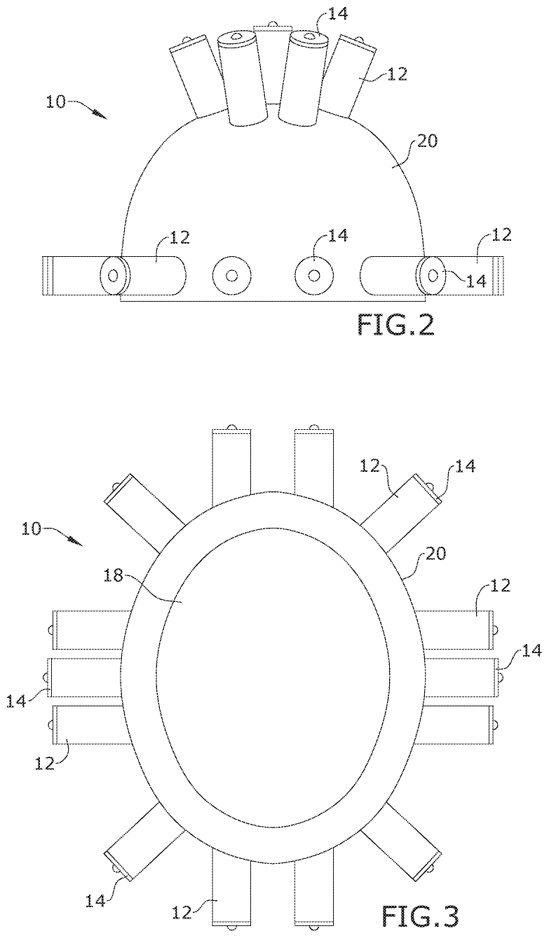

Referring now to the FIGS. 1-3, a helmet 10 comprises channels 12 extending radially from the helmet 10. Plugs 14 fit into the channels 12. The helmet 10 includes a cavity 18 for a user 16 to place their head. A plastic layer 20 is on an exterior of the helmet 10.

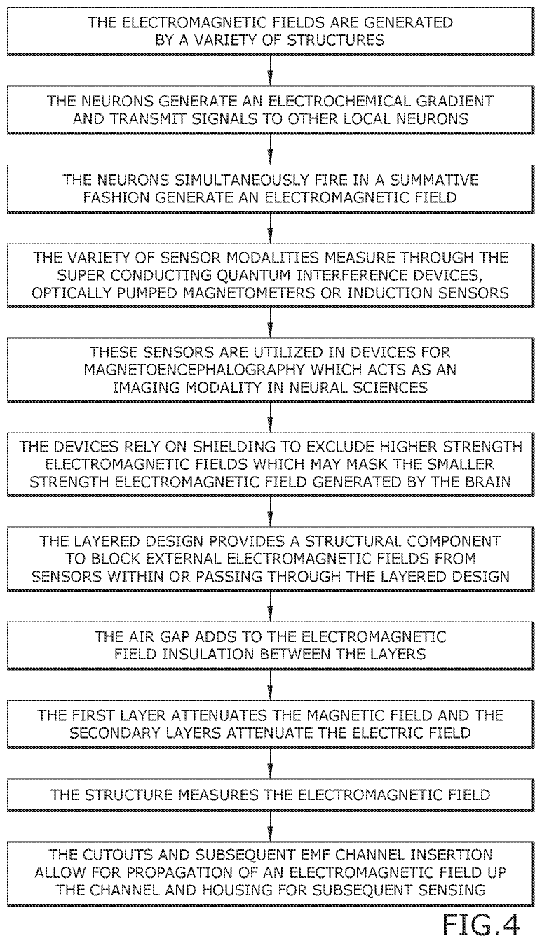

FIG. 4 describes a process in which electromagnetic fields are generated and the present invention's relationship to said fields. A layered design of the present invention blocks external electromagnetic fields from sensors within the helmet 10, preventing electromagnetic fields from passing through the present invention. An air gap in the helmet 10 adds electromagnetic field insultation. Layers of the helmet 10 attenuate the magnetic field and the electric field. The channels 12 and plugs 14 enables propagation of electromagnetic fields through the helmet 10, through respective channels 12. The channels may be closed or blocked by plugs 14.

As used in this application, the term “about” or “approximately” refers to a range of values within plus or minus 10% of the specified number or value. And the term “substantially” refers to up to 80% or more of an entirety. Recitation of ranges of values herein are not intended to be limiting, referring instead individually to any and all values falling within the range, unless otherwise indicated, and each separate value within such a range is incorporated into the specification as if it were individually recited herein.

For purposes of this disclosure, the term “aligned” means parallel, substantially parallel, or forming an angle of less than 35.0 degrees. For purposes of this disclosure, the term “transverse” means perpendicular, substantially perpendicular, or forming an angle between 55.0 and 125.0 degrees. Also, for purposes of this disclosure, the term “length” means the longest dimension of an object. Also, for purposes of this disclosure, the term “width” means the dimension of an object from side to side. For the purposes of this disclosure, the term “above” generally means superjacent, substantially superjacent, or higher than another object although not directly overlying the object. Further, for purposes of this disclosure, the term “mechanical communication” generally refers to components being in direct physical contact with each other or being in indirect physical contact with each other where movement of one component affect the position of the other.

The use of any and all examples, or exemplary language (“e.g.,” “such as,” or the like) provided herein, is intended merely to better illuminate the embodiments and does not pose a limitation on the scope of the embodiments or the claims. No language in the specification should be construed as indicating any unclaimed element as essential to the practice of the disclosed embodiments.

In the following description, it is understood that terms such as “first,” “second,” “top,” “bottom,” “up,” “down,” and the like, are words of convenience and are not to be construed as limiting terms unless specifically stated to the contrary.

It should be understood, of course, that the foregoing relates to exemplary embodiments of the invention and that modifications may be made without departing from the spirit and scope of the invention as set forth in the following claims.

What is claimed is:

1. A device for shielding of electromagnetic fields comprising:
a helmet comprising layers of a nickel-iron ferromagnetic alloy, a copper mesh, and an air gap;
at least one channel extending radially from the helmet;
at least one plug configured to removably fit into respective channels wherein each plug is operative shielding electromagnetic fields by way of the channel; and
at least one sensor incorporated into the helmet.

2. The device of claim 1, wherein the helmet operates by a method comprising:
providing shielding from an external electromagnetic field;
containing an electromagnetic field on an interior of the helmet; and
funneling the electromagnetic field on the interior of the helmet to the at least one sensor.

3. The device of claim 2, further comprising identifying generated electromagnetic fields by the at least one sensor.

4. The device of claim 2, further comprising operatively associating the at least one plug into the at least one channel, respectively, to shield electromagnetic fields travelling through said channel.

5. The device of claim 1, wherein the at least one sensor is incorporated into the at least one channel.

6. The device of claim 1, wherein each plug is composed of layers of a nickel-iron ferromagnetic alloy, a copper mesh, and an air gap.

7. The device of claim 1, wherein the helmet is configured to fit on a user's head.
